(19) United States
(12) Patent Application Publication
(10) Pub. No.: US 2010/0261284 A1
(43) Pub. Date: Oct. 14, 2010

(54) USING GDF 15 TO ASSESS PATIENTS PRESENTING TO EMERGENCY UNITS

(76) Inventor: Eberhard Spanuth, Dossenheim (DE)

Correspondence Address: ROCHE DIAGNOSTICS OPERATIONS INC., 9115 Hague Road, Indianapolis, IN, 46250-0457, US

(21) Appl. No.: 12/822,690

(22) Filed: Jun. 24, 2010

(30) Foreign Application Priority Data
Jan. 8, 2008 (EP) 08150098.5
Jan. 8, 2009 (EP) PCT/EP2009/050170

(51) Int. Cl. G01N 33/68

(52) U.S. Cl. 436/86; 422/68.1

(57) ABSTRACT

Described is a method of identifying if a subject is to be admitted to the hospital or intensive care unit, the method comprising a) determining the amount of GDF 15 in a sample of the subject, and b) comparing the amount of GDF 15 determined in step a) to a reference amount, whereby a subject to be admitted to the hospital or intensive care unit is to be identified. Also described is a method for predicting the risk of mortality based on determining the amount of GDF 15 in a subject. Also described are devices and kits for carrying out the aforementioned methods.

### RELATED APPLICATIONS

This application is a continuation of International application PCT/EP2009/050170 filed Jan. 8, 2009 and claims priority to European application EP 08150098.5 filed Jan. 18, 2008.

### FIELD OF THE INVENTION

The present invention relates to a method of risk stratification in a subject. The method according to the present invention permits identifying if a subject is to be admitted to the hospital or intensive care unit or can be discharged to home. In most cases, the subject presents to the emergency unit. The method is based on the determination of growth differentiation factor 15 (GDF 15) in a sample of the subject. Also encompassed by the present invention are devices and kits for carrying out the aforementioned methods.

### BACKGROUND OF THE INVENTION

When subjects present to an emergency unit with any kind of discomfort, a rapid diagnosis of the pathological state of the subject is mandatory in order to identify the cause underlying his discomfort and avoid consequences to the subject's health. A highly relevant topic is the decision if the patient will be admitted to the hospital—for further, time consuming analysis and/or intensive care treatment, or if the patient can be discharged to home.

For example, in the case of acute cardiovascular events, a decision for a certain treatment regimen must be made, usually, within a short period of time. Cardiovascular complications, particularly heart diseases, are the leading cause of morbidity and mortality in the Western hemisphere. Cardiovascular complications can remain asymptomatic for long periods of time. However, they may have severe consequences once an acute cardiovascular event, such as myocardial infarction, as a cause of the cardiovascular complication occurs. Therefore, guidelines exist for the rapid diagnosis of patients presenting to a physician, generally in an emergency unit, and being suspected of suffering from an acute coronary syndrome ACS (i.e., unstable angina pectoris UAP or myocardial infarction MI), see J. Am. Coll. Cardiol. 2000; 36, pages 959-969. In a subject suspected to have MI, an electrocardiogram is recorded, and the level of Troponin T or I is determined. Further, it is analyzed if the suspected subject shows evident syndromes like chest pain, palpitation, nausea, vomiting and further syndromes known to the person skilled in the art. If the subject is positive in 2 of the 3 criteria, then he or she is admitted to the hospital for further examination.

However, the composition of the subjects presenting to an emergency unit is heterogeneous, comprising about 35% of subjects suffering from cardiovascular complications (including both acute, i.e., ischemic complications and non-ischemic complications), 10% of subjects suffering from pulmonary complications and 55% or 56% subjects suffering from other complications, e.g., tumors, and which may additionally suffer from cardiovascular complications. The conventional diagnostic techniques, specifically for emergency situations, usually do not allow for a reliable diagnosis and/or risk assessment covering these various pathological states. A further drawback is the often occurring lack of personnel and the varying occupancy in emergency units.

At present, there does not exist a standardized diagnosis procedure covering the various diseases a physician may encounter in an emergency unit. Thus, a rapid and accurate diagnosis allowing a decision if the subject can be discharged to home or has to be admitted to the hospital for further examination or intensive care treatment (which can be live saving) cannot be carried out in the emergency unit with sufficient accuracy. As a consequence thereof, many patients will either be admitted or discharged in to home where the opposite would have been the appropriate measure.

In some cases, so-called molecular markers permit to establish rapid and sufficiently accurate diagnosis of the pathological state of a subject. A prominent example is troponin T and/or troponin I for the diagnosis of MI, as mentioned beforehand, or natriuretic peptides, in particular NT-proBNP for various non-ischemic heart diseases, e.g., heart failure.

Recently, GDF 15 has been suggested to be an indicator for cardiovascular complications, too (US2003/0232385; Kempf 2006, Circ Res 98: 351-360). Growth-differentiation factor-15 (GDF 15) is a member of the transforming growth factor-βcytokine superfamily. GDF 15 was first identified as macrophage-inhibitory cytokine-1 (MIC-1), and later also named placental transforming growth factor-β(Bootcov 1997, Proc Natl Acad Sci 94:11514-11519; Tan 2000, Proc Natl Acad Sci 97:109-114). It has recently been shown that cultured cardiomyocytes express and secrete GDF 15 via nitric oxide and nitrosative stress-dependent signaling pathways when subjected to simulated ischemia and reperfusion. Moreover, it has been observed in a mouse model of myocardial ischemia and reperfusion injury that GDF 15 expression levels rapidly increase in the ischemic area following coronary artery ligation, and remain elevated in the reperfused myocardium for several days (Kempf loc. cit.).

The application PCT/EP2007/058007 filed Aug. 2, 2007, relates to a method of identifying a subject being susceptible to a cardiac intervention based on the determination of GDF 15 in a sample of a subject in need of a cardiac intervention. Moreover, the invention pertains to a method for predicting the risk of mortality or a further acute cardiovascular event for a subject suffering from a cardiovascular complication based on the determination of GDF 15 and a natriuretic peptide and/or a cardiac Troponin in a sample the subject.

Therefore, there is a need for diagnostic or prognostic measures which allow an assessment and/or an individual risk stratification for a subject presenting to the emergency unit. The measures should permit this assessment/risk stratification also in patients not presenting with cardiovascular diseases, in particular not with acute cardiovascular diseases.

The technical problem underlying the present invention can be seen as the provision of means and methods for complying with the aforementioned needs.

The technical problem is solved by the embodiments characterized in the claims and herein below.

### SUMMARY OF THE INVENTION

Accordingly, the present invention relates to a method of identifying if a subject is to be admitted to the hospital, the method comprising
a) determining the amount of GDF 15 in a sample of the subject; and
b) comparing the amount of GDF 15 determined in step a) to a reference amount, whereby a subject to be admitted to the hospital is to be identified.

### BRIEF DESCRIPTION OF THE DRAWINGS

FIG. 1 shows admission and discharge rates depending on GDF 15 values above or below the GDF 15 median value. Patients with GDF 15 values above the median of 1,6605 ng/ml were admitted more often (Admission/Discharge rate=2.17) compared to those with GDF 15 values below the median (Admission/Discharge rate=0.53).

The quartile ranges are displayed in FIG. 2 showing that patients with increasing GDF 15 values were admitted to the hospital. Patients in the 1st quartile revealed an admission/discharge rate of 0.43 compared with an admission/discharge rate of 4.85 of patients in the 4th quartile.

Results of ROC analysis for prediction discharge or admission by using GDF 15 values are shown in FIG. 3. The area under the curve (AUC) of 0.722 demonstrates the high discrimination power of GDF 15 between discharge and admission. The GDF 15 cut off value with optimal sensitivity/negative predictive value (66.7%) and optimal specificity/positive predictive value (68.6%) was estimated as 1.56 ng/ml.

### DETAILED DESCRIPTION OF THE INVENTION

In general, the subject presents to a physician, often a general practitioner. Preferably, the subject is a subject presenting to an emergency unit, preferably in a hospital, even more preferably an internistic emergency unit.

In the context of the present invention, the term “emergency unit” refers to any location where individuals feeling uncomfortable present, in order to consult a person having a medical background, preferably a physician, to have an analysis of their pathological state and the cause underlying their discomfort. Typical examples are emergency departments or emergency rooms in hospitals, emergency ambulances, doctor's offices and other institutions suitable for treatment of critical ill patients.

In the context of the present invention, the term “hospital” refers to any location where individuals which have a disease or are suspected of having a disease are taken up or kept, in order to be further diagnosed, watched, treated or taken care of. “Hospital” thus refers to hospitals as such, and includes any units therein, including normal care units. In a preferred embodiment of the present invention, the hospital unit is an intensive care unit. Therefore, in one embodiment, the method according to the present invention allows to identify subjects which are to be admitted to the intensive care unit, as a consequence of their physiological condition requiring more than a normal care unit.

The method of the present invention, preferably, is an in vitro method. Moreover, it may comprise steps in addition to those explicitly mentioned above. For example, further steps may relate to sample pre-treatments or evaluation of the results obtained by the method. The method may be carried out manually or assisted by automation. Preferably, step (a) and/or (b) may in total or in part be assisted by automation, e.g., by a suitable robotic and sensory equipment for the determination in step (a) or a computer-implemented comparison in step (b).

The term “identifying” as used herein means assessing whether a subject is in a pathological state necessitating admission to the hospital for further examination, and/or intensive care treatment, or intervention. As will be understood by those skilled in the art, such an assessment is usually not intended to be correct for all (i.e., 100%) of the subjects to be identified. The term, however, requires that a statistically significant portion of subjects can be identified (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method of the present invention.

The term “subject” as used herein relates to animals, preferably mammals, and, more preferably, humans.

In accordance with the present invention, the subject suffers from discomfort and/or a pathological state unknown to him and which he cannot diagnose on his own, and feels that a physician should be consulted. The subject is an average subject, i.e., belonging to the average subject population as disclosed beforehand (about 35% suffering from cardiovascular complications, 10% suffering from pulmonary complications and 55% or 56% suffering from other complications, e.g., tumors, and may additionally suffer from cardiovascular complications).

However, it is envisaged in accordance with the aforementioned method of the present invention that the subject shall not be suffering from a complication which can be diagnosed by following a standard diagnosis protocol, preferably an acute cardiovascular event as defined by the American College of Cardiology (see above), e.g., chest discomfort, dyspnea, ECG changes and others as described above. More preferably, the subject shall not exhibit one or more episodes of angina lasting at least 5 min within the preceding 24 h, and not have either a positive cardiac troponin T or I test or at least 0-5 mm of transient or persistent ST-segment depression not known to be preexisting and not attributable to coexisting disorders. Alternatively, the subject shall not exhibit symptoms of ischemia that were increasing or occurring at rest, or that warranted the suspicion of acute myocardial infarction, with the last episode within the preceding 48 h. Myocardial ischemia has to be verified by electrocardiography (ST depression=0.1 mV or T-wave inversion=0.1 mV) or by raised biochemical markers (creatine kinase [CK]-MB>6 ug/L, troponin-T>0.01 ng/ml, qualitative troponin-T test positive, or catalytic activity of CK, CK-B, or CK MB higher than the local diagnostic limit for myocardial infarction).

Acute cardiovascular events are, preferably, acute coronary syndromes (ACS). ACS patients can show unstable angina pectoris (UAP) or myocardial infarction (MI). MI can be an ST-elevation MI (STEMI) or a non-ST-elevated MI (NSTEMI). The occurring of an ACS can be followed by a left ventricular dysfunction (LVD) and symptoms of heart failure.

In accordance with the foregoing, the method of the present invention can also be described as being a method of diagnosing or assessing if an individual having a disease or being suspected of having a disease has to be further diagnosed, watched, treated or taken care of, preferably in a hospital.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

The term “Growth-Differentiation Factor-15” or “GDF 15” relates to a polypeptide being a member of the transforming growth factor (TGF)-β cytokine superfamily. The terms polypeptide, peptide and protein are used interchangeable throughout this specification. GDF 15 was originally cloned as macrophage-inhibitory cytokine-1 and later also identified as placental transforming growth factor-β, placental bone morphogenetic protein, non-steroidal anti-inflammatory drug-activated gene-1, and prostate-derived factor (Bootcov loc cit; Hromas, 1997 Biochim Biophys Acta 1354:40-44; Lawton 1997, Gene 203:17-26; Yokoyama-Kobayashi 1997, J Biochem (Tokyo), 122:622-626; Paralkar 1998, J Biol Chem 273:13760-13767). Similar to other TGF-β-related cytokines, GDF 15 is synthesized as an inactive precursor protein, which undergoes disulfide-linked homodimerization. Upon proteolytic cleavage of the N-terminal pro-peptide, GDF 15 is secreted as a ˜28 kDa dimeric protein (Bauskin 2000, Embo J 19:2212-2220). Amino acid sequences for GDF 15 are disclosed in WO99/06445, WO00/70051, WO2005/113585, Bottner 1999, Gene 237: 105-111, Bootcov loc. cit, Tan loc. cit., Baek 2001, Mol Pharmacol 59: 901-908, Hromas loc cit, Paralkar loc cit, Morrish 1996, Placenta 17:431-441 or Yokoyama-Kobayashi loc cit. GDF 15 as used herein encompasses also variants of the aforementioned specific GDF 15 polypeptides. Such variants have at least the same essential biological and immunological properties as the specific GDF 15 polypeptides. In particular, they share the same essential biological and immunological properties if they are detectable by the same specific assays referred to in this specification, e.g., by ELISA assays using polyclonal or monoclonal antibodies specifically recognizing the GDF 15 polypeptides. A preferred assay is described in the accompanying examples. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific GDF 15 polypeptides. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Moreover, the variants referred to herein include fragments of the specific GDF 15 polypeptides or the aforementioned types of variants as long as these fragments have the essential immunological and biological properties as referred to above. Such fragments may be, e.g., degradation products of the GDF 15 polypeptides. Further included are variants which differ due to posttranslational modifications such as phosphorylation or myristylation.

Determining the amount of GDF 15 or any other peptide or polypeptide referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR—analyzers, or chromatography devices. Further, methods include micro-plate ELISA-based methods, fully-automated or robotic immunoassays (available for example on ELECSYS analyzers, Roche Diagnostics GmbH), CBA (an enzymatic cobalt binding assay, available, for example, on Roche/Hitachi analyzers), and latex agglutination assays (available for example on Roche/Hitachi analyzers).

Preferably, determining the amount of a peptide or polypeptide comprises the steps of (a) contacting a cell capable of eliciting a cellular response the intensity of which is indicative of the amount of the peptide or polypeptide with the peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprises the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)₂ fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g. the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for an detectable, preferably measurable, amount of product to be produced. Instead of measuring the amount of product, the time necessary for appearance of a given (e.g. detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxigenin, His-Tag, Glutathione-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g., horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star (Amersham Biosciences), ECF (Amersham Biosciences). A suitable enzyme-substrate combination may result in a colored reaction product, fluorescence or chemiluminescence, which can be measured according to methods known in the art (e.g. using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, fluorescein, and the Alexa dyes (e.g., Alexa 568). Further fluorescent labels are available, e.g., from Molecular Probes (Oregon). Also the use of quantum dots as fluorescent labels is contemplated. Typical radioactive labels include 35S, 125I, 32P, 33P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoroimmunoassay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting, and mass spectrometry), can be used alone or in combination with labeling or other detection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the polypeptide or peptide as well as any value or parameter which correlates thereto or can be derived therefrom. Such values or parameters comprise intensity signal values from all specific physical or chemical properties obtained from the peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (b) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e., automatically provide the desired assessment in a suitable output format. Based on the comparison of the amount determined in step a) and the reference amount, it is possible to assess whether a subject is susceptible for a cardiac intervention, i.e., belonging to the group of subjects which can be successfully treated by the cardiac intervention. Therefore, the reference amount is to be chosen so that either a difference or a similarity in the compared amounts allows identifying those the test subject which belong into the group of subjects susceptible for cardiac intervention or identifying those test subjects which are not susceptible for a cardiac intervention.

Accordingly, the term “reference amount” as used herein refers to an amount which allows assessing whether a subject is to be admitted to the hospital or can be discharged to home. Accordingly, the reference may e.g., be derived from (i) a subject known to have been successfully admitted to the hospital, i.e., who has been subject to further examination and subsequent or intensive care treatment based on the results of the further investigation without the occurrence of adverse effects such as mortality or side effects caused by unadapted treatment regimen, or (ii) a subject known to have not been admitted to the hospital and which died or developed side effects caused by unadapted treatment regimen. Moreover, the reference amount may define a threshold amount, whereby an amount larger than the threshold shall be indicative for a subject which should be admitted to the hospital for further examination and/or (intensive) treatment, while an amount lower than the threshold amount shall be an indicator for a subject which can be discharged to home. The reference amount applicable for an individual subject may vary depending on various physiological parameters such as age, gender, or subpopulation, as well as on the means used for the determination of the polypeptide or peptide referred to herein. A suitable reference amount may be determined from a reference sample to be analyzed together, i.e., simultaneously or subsequently, with the test sample. A preferred reference amount serving as a threshold may be derived from the upper limit of normal (ULN), i.e., the upper limit of the physiological amount to be found in a population of apparently healthy subjects. The ULN for a given population of subjects can be determined by various well known techniques. A suitable technique may be to determine the median of the population for the peptide or polypeptide amounts to be determined in the method of the present invention. A preferred threshold (i.e. reference amount) for GDF 15 is at least one to two times the ULN. The ULN referred to in this context is, preferably, 1800 pg/ml

Thus, the reference amount defining a threshold amount for GDF 15 as referred to in accordance with the present invention is 1200 pg/ml, preferably 1560 pg/ml and, most preferably, 8210 pg/ml (representing a negative predictive value of 99.3%) for admission to hospital.

An amount of GDF 15 larger than the reference amount is, more preferably, indicative for a subject which should be admitted to hospital.

In addition or alternatively, the above method of the present invention may be used to identify a subject susceptible to intensive care treatment in an intensive care unit. The intensive care treatment may comprise additional diagnostic procedures and therapeutic interventions. A therapeutic intervention may be a drug based therapy or comprises all kinds of surgical interventions.

Additional diagnostic procedures include: transthoracal echocardiography; transesophageal echocardiography; abdominal sonographie; CT (thorax); x-ray (thorax); spiral CT; invasive cardiac catheterization diagnostic (left, right, combined); lung scintigraphic (inhalation and perfusion); compression sonographie of legs; stress echocardiography; bronchoscopy; phlebography; angiography.

Drugs which may be administered include: oral anticoagulants, unfractionated heparins and other antithrombins and fibrinolytic agents; ASS; clopidrogel; loop diuretics and other diuretics; beta blockers; ACE inhibitors; AT blockers; digitalis; calcium antagonists; nitrates; steroids (oral and inhalation); theophyllin; beta sympathomimetics and other bronchodilators; opiates; antibiotics.

It is to be understood that the definitions and explanations of the terms made above and below apply accordingly for all embodiments described in this specification and the accompanying claims.

The present invention further relates to a method of deciding about admitting a subject to the hospital, the method comprising
a) determining the amount of GDF 15 in a sample of the subject; and
b) comparing the amount of GDF 15 determined in step a) to a reference amount; and
c) deciding whether the subject is to be admitted to the hospital.

Preferably, the subject is a subject presenting to the emergency unit. In the hospital, the subject may be admitted to the intensive care unit.

Another preferred therapy to be selected for a subject in accordance with the present invention is an interventional therapy. An interventional therapy as referred to herein is a therapy which is based on physical interventions with the subject, e.g., by surgery.

The subject in this embodiment of the invention is the same subject as defined in the earlier embodiments, in particular a subject not suffering from an acute cardiovascular event.

The present invention, furthermore, relates to a method for predicting the risk of mortality for a subject comprising
a) determining the amount of GDF 15 in a sample of the subject; and
b) comparing the amount of GDF 15 determined in step a) to a reference amount; and
c) predicting the risk of mortality based on the result of steps a) and b).

Preferably, the subject is a subject presenting to the emergency unit. In the hospital, the subject may be admitted to the intensive care unit.

The subject in this embodiment of the invention is the same subject as defined in the earlier embodiments, in particular a subject not suffering from an acute cardiovascular event.

The term “predicting” used herein refers to assessing the probability according to which a subject suffering from a disease will die within a defined time window (predictive window) in the future. The predictive window is an interval in which the subject will die according to the predicted probability. The predictive window may be the entire remaining lifespan of the subject upon analysis by the method of the present invention. Preferably, however, the predictive window is an interval of one month, six months or one, two, three, four, five or ten years after appearance of the cardiovascular complication (more preferably and precisely, after the sample to be analyzed by the method of the present invention has been obtained). As will be understood by those skilled in the art, such an assessment is usually not intended to be correct for 100% of the subjects to be analyzed. The term, however, requires that the assessment will be valid for a statistically significant portion of the subjects to be analyzed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the prediction will be correct for at least 60%, at least 70%, at least 80%, or at least 90% of the subjects of a given cohort.

The term “mortality” as used herein relates to mortality which is caused by cardiovascular complications, lung diseases, lung embolism, thrombosis, thromboembolic complications, stroke, malignant diseases, sepsis, septic shock, bleeding disorders, organ failure, acute kidney disease, and others.

The term “cardiovascular complication” as used herein refers to any chronic disorder of the cardiovascular system or any acute cardiovascular event. Preferably, a chronic disorder of the cardiovascular system as used herein encompasses coronary heart diseases, stable angina pectoris (SAP) or heart failure, preferably chronic heart failure. Acute cardiovascular events are, preferably, acute coronary syndromes (ACS). ACS patients can show unstable angina pectoris (UAP) or myocardial infarction (MI). MI can be an ST-elevation MI (STEMI) or a non-ST-elevation MI (NSTEMI). NSTE-ACS as used herein encompasses UAP and NSTEMI. The occurring of an MI can be followed by a left ventricular dysfunction (LVD) or development of heart failure. Further preferred cardiovascular complications encompass cardiac brady- or tachyarrhythmias including sudden cardiac death and stroke (cerebrovascular events or accidents). Most preferably, the cardiovascular complication is ACS or heart failure.

The expression “predicting the risk of mortality” as used herein means that it the subject to be analyzed by the method of the present invention is allocated either into the group of subjects of a population having a normal, i.e., non-elevated, risk for mortality or into a group of subjects having a significantly elevated risk. An elevated risk as referred to in accordance with the present invention means that the risk of mortality within a predetermined predictive window is elevated significantly for a subject with respect to the average risk for mortality in a population of subjects. Preferably, for a predictive window of one year, the average risk is within the range of 0.5 and 3.0%, preferably, 1.5%. An elevated risk as used herein, preferably, relates to a risk of more than 3.0%, preferably, more than 5.0%, and, most preferably within 3.0% and 8.0% with respect to a predictive window of one year.

Encompassed by the present invention is, further, a device for identifying a subject to be admitted to the hospital, adapted to carry out the method of the present invention, comprising means for determining the amount of GDF 15 in a sample of the subject and means for comparing said amount to a reference amount, whereby a subject to be admitted to the hospital is identified.

Preferably, the subject is a subject presenting to the emergency unit. In the hospital, the subject may be admitted to the intensive care unit.

The term “device” as used herein relates to a system of means comprising at least the aforementioned means operatively linked to each other as to allow the prediction. Preferred means for determining the amount of GDF 15 and means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of said peptides or polypeptides to the ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e., evaluated, raw data the interpretation of which does not require a clinician. Further preferred devices comprise the analyzing units/devices (e.g., biosensors, arrays, solid supports coupled to ligands specifically recognizing the natriuretic peptide, Plasmon surface resonance devices, NMR spectrometers, mass-spectrometers etc.) or evaluation units/devices referred to above in accordance with the method of the invention.

Moreover, the present invention also relates to a device for predicting the risk of mortality or a further acute cardiovascular event for a subject adapted to carry out the method of the present invention comprising means for determining the amount of GDF 15 and means for comparing said amounts to reference amounts, whereby it is predicted whether a subject is at risk of mortality or a further acute cardiovascular event.

Further envisaged is a device for deciding about admitting a subject to the hospital, adapted to carry out the method of the present invention, comprising means for determining the amount of GDF 15 in a sample of the subject and means for comparing said amount to a reference amount, whereby it is decided whether the subject is to be admitted to the hospital.

The present invention also relates to a device for predicting the risk of mortality in a subject, adapted to carry out the method of the present invention, comprising means for determining the amount of GDF 15 in a sample of the subject and means for comparing said amounts to reference amounts, whereby it is predicted whether the subject is at risk of mortality.

Preferably, the subject is a subject presenting to the emergency unit. In the hospital, the subject may be admitted to the intensive care unit.

Furthermore, a kit for carrying out the methods of the present invention, for identifying a subject to be admitted to the hospital, deciding about admitting a subject to the hospital, or predicting the risk of mortality in a subject is envisaged by the present invention. Said kit comprising means for determining the amount of GDF 15 in a sample of a subject and means for comparing said amounts to reference amounts, wherein a subject to be admitted to the hospital is identified, a decision about admitting the subject to the hospital or intensive care unit is made, or the risk of mortality in the subject is predicted.

The term “kit” as used herein refers to a collection of the aforementioned means, preferably, provided in separately or within a single container. The container, also preferably, comprises instructions for carrying out the method of the present invention.

All references cited in this specification are herewith incorporated by reference with respect to their entire disclosure content and the disclosure content specifically mentioned in this specification.

### EXAMPLE 1

We tested the hypothesis that use of GDF 15 improves decision making in the emergency room and the overall management of patients presenting to emergency departments (EDs) by evaluating for incremental diagnostic and prognostic value of GDF 15, and prospectively examining the clinical impact of GDF 15 guided decision making regarding discharge, admission to hospital, and intensive care treatment.

### Methods:

A total of 303 patients presenting to the emergency department (ED) of an university hospital were studied. Blood samples were obtained in the ED from all patients admitted. GDF 15 was determined in 302 unselected consecutive patients. Follow-up at discharge included the assessment of clinical course and treatment. The variables discharge, admission, and intensive care treatment were studied and associated to the baseline GDF 15 values. Cut-off thresholds of GDF 15 suitable for risk stratification and medical decision making (discharge or admissions to ICU or general care units), were calculated using ROC analysis.

### Results:

Presenting diagnoses were grouped into 4 categories:
1. Confirmed or suspected ischemic heart disease, including acute coronary syndrome and
ischemic heart failure: 33 (10.9%)
2. Nonischemic heart disease, including arrhythmias without acute ischemic trigger, vascular heart disease, and cardiomyopathies: 36 (11.8%)
3. Lung disorders, including asthma, chronic obstructive disease, and pulmonary embolism: 41 (21.8%)
   4. All other disorders: 193 (63.5%)

In summary, 63.5% of the patients presented at the ED with non-cardiovascular or non-respiratory disorders whereas 22.7% presented with ischemic and non-ischemic heart diseases.

### Statistical Analysis

### 1. Descriptive Statistic

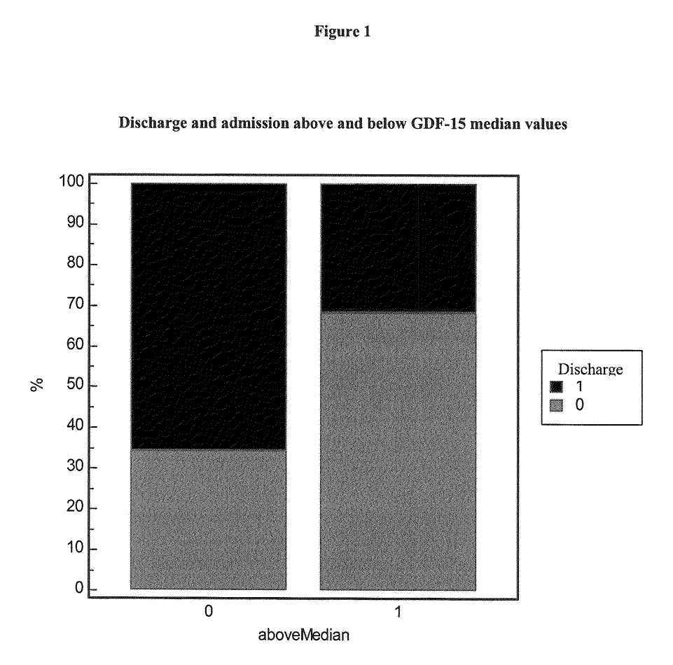

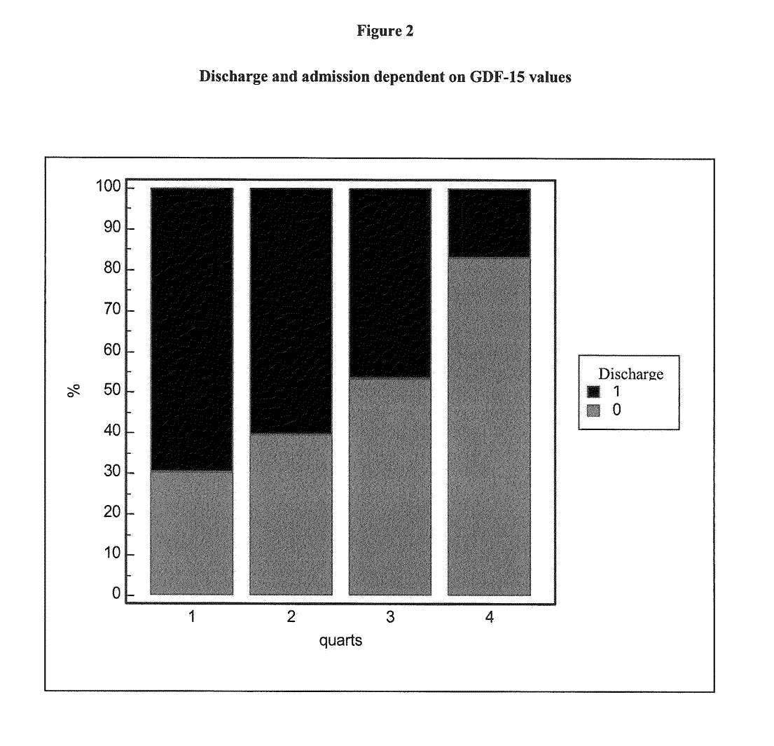

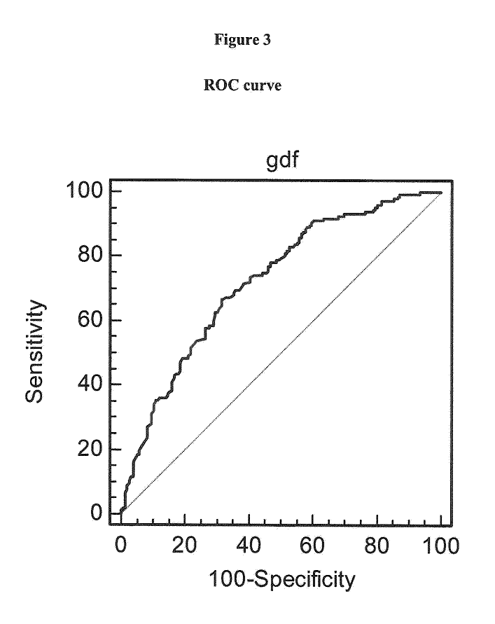

Table 1 shows the values of arithmetic mean, median, and percentils and Table 2 and FIG. 1 show admission and discharge rates depending on GDF 15 values above or below the GDF 15 median value. Patients with GDF 15 values above the median of 1,6605 ng/ml were admitted more often (Admission/Discharge rate=2.17) compared to those with GDF 15 values below the median (Admission/Discharge rate=0.53).

### 2. Quartiles

The quartile ranges are displayed in Table 3 and FIG. 2 showing that patients with increasing GDF 15 values were admitted to the hospital. Patients in the 1rst quartile revealed an admission/discharge rate of 0.43 compared with an admission/discharge rate of 4.85 of patients in the 4th quartile.

### 3. ROC Analysis

Results of ROC analysis for prediction discharge or admission by using GDF 15 values are shown in Table 4. The area under the curve (AUC) of 0.722 demonstrates the high discrimination power of GDF 15 between discharge and admission. The GDF 15 cut off value with optimal sensitivity/negative predictive value (66.7%) and optimal specifity/positive predictive value (68.6%) was estimated as 1.56 ng/ml.

4. “Rule in” GDF 15 Cut Off Value for Admission

In order to identify patients presenting at the emergency department who have to be admitted to hospital a GDF 15 “rule in” cut off value of 8.21 ng/ml could be obtained from the ROC analysis. The corresponding sensitivity and negative predictive value is 99.3% and 95.2%, respectively. Patients with GDF 15 values >8.21 ng/ml should be admitted to hospital (and not be discharged) independent of diagnosis and underlying disease.

### CONCLUSION

GDF 15 is a marker for decision making and a suitable tool for risk stratification of patients presenting at the emergency department. Moreover, by using GDF 15 “rule in” cut off value patients could be identified who should be admitted to hospital and not be discharged independent of diagnosis and disease status of the patient.

TABLE 1
Descriptive statistic variables

| | GDF15 | |
|---|---|---|
| Sample size | 303 | |
| Lowest value | 0.2953 | |
| Highest value | 57.8772 | |
| Arithmetic mean | 3.2219 | |
| 95% CI for the mean | 2.6235 to 3.8202 | |
| Median | 1.6605 | |
| 95% CI for the median | 1.4963 to 1.8185 | |
| Variance | 28.0115 | |
| Standard deviation | 5.2926 | |
| Relative standard deviation | 1.6427 | (164.27%) |
| Standard error of the mean | 0.3041 | |
| Coefficient of Skewness | 6.0022 | (P < 0.0001) |
| Coefficient of Kurtosis | 48.5726 | (P < 0.0001) |
| D'Agostino-Pearson test | reject Normality | (P < 0.0001) |
| for Normal distribution | | |

| Percentiles | | 95% Confidence Interval |
|---|---|---|
| 2.5 | 0.5306 | 0.4614 to 0.6417 |
| 5 | 0.6411 | 0.5363 to 0.7423 |
| 10 | 0.7661 | 0.7092 to 0.8363 |
| 25 | 1.0985 | 0.9755 to 1.2030 |
| 75 | 3.0278 | 2.5473 to 4.1356 |
| 90 | 6.1868 | 5.2472 to 8.6704 |
| 95 | 10.8089 | 7.3520 to 15.8687 |
| 97.5 | 15.9309 | 10.6962 to 27.9267 |

TABLE 2
Fisher's exact test

| | Classification X | | |
|---|---|---|---|
| Classification Y | GDF 15 < Median | GDF 15 > Median | |
| Admission | 52 | 104 | 156 |
| Discharge | 99 | 48 | 147 |
| | 151 | 152 | 303 |

Classification X aboveMedian
Classification Y Discharge
P = 0.000000003

TABLE 3
Frequency table & Chi-square test

| | |
|---|---|
| Codes X | quarts |
| Codes Y | Discharge |

Codes X

| | | | | |
|---|---|---|---|---|
| Codes Y | 1 | 2 | 3 | 4 |

| | | | | | |
|---|---|---|---|---|---|
| Admission | 23 | 30 | 40 | 63 | 156 |
| Discharge | 53 | 46 | 35 | 13 | 147 |
| Admission/Discharge | 0.43 | 0.65 | 1.14 | 4.85 | |
| rate | | | | | |

| | |
|---|---|
| Chi-square | 48,214 |
| DF | 3 |
| Significance level | P < 0.0001 |

Chi-square test for trend

| | |
|---|---|
| Chi-square (trend) | 44,896 |
| DF | 1 |
| Significance level | P < 0.0001 |

### EXAMPLE 2

A 58 years old female was admitted to the emergency room with suspected gastrointestinal bleeding or acute gastritis.

### Case History:

Chronic hepatitis B, liver cirrhosis

### Physical Examination:

Weight: 62 Kg
Size: 162 cm
Heart rate: 60/min
Blood pressure: 70/138 mmHg

### Clinical Signs and Symptoms:

No clinical signs for cardiovascular impairment.
No clear clinical signs related to gastrointestinal bleeding or gastritis.
   Laboratory:
GDF 15 value: 1.56 ng/ml; <8.21 ng/ml (rule in cut off value for admission to hospital); <1.66 ng/ml (optimized ROC cut off value)

### Result:

The patient could be discharged to home!

### EXAMPLE 3

A 81 years old male was admitted to the emergency room with suspected myocardial infarction in an unclear clinical situation.

### Case History:

Stable coronary heart disease, kidney disease

### Physical Examination:

Weight: 100 Kg
Size: 178 cm
Temperature: 39° C.
Heart rate: 103/min
Blood pressure: 70/138 mmHg

### Clinical Signs and Symptoms:

Dyspnea
Cardiac murmur but no clinical signs of decompensated acute heart failure.
No signs of myocardial infarction.

### Laboratory:

GDF 15 value: 20.62 ng/ml; >8.21 ng/ml (rule in cut off value for admission to hospital).

### Result:

The patient was admitted to the intensive care unit.

### Example 4

A 27 years old male was admitted to the emergency department with suspected acute gastritis.

### Case History:

Patient has never been hospitalized, and takes no medication. She exhibited no signs of health problems.

### Physical Examination:

Weight: 85 Kg
Size: 180 cm
Temperature: 36° C.
Heart rate: 65/min
Blood pressure: 85/126 mmHg

### Clinical Signs and Symptoms:

Tickle/itch paresthesia
Hyperventilation

### Laboratory:

GDF 15 value: 0.53 ng/ml; <8.21 ng/ml (rule in cut off value for admission to hospital); <1.66 ng/ml (optimized ROC cut off value)

### Result:

The patient was discharged to home.

### EXAMPLE 5

A 37 years old female was admitted to the emergency room with vasovagale syncope.

### Physical Examination:

Weight: 98 Kg
Size: 167 cm
Temperature: 37.2° C.
Heart rate: 84/min
Blood pressure: 90/140 mmHg

### Clinical Signs and Symptoms:

Chest pain, atypical for myocardial infarction
No other signs of cardiovascular disease

### Laboratory:

GDF 15 value: 0.91 ng/ml; <8.21 ng/ml (rule in cut off value for admission to hospital); <1.66 ng/ml (optimized ROC cut off value)

### Result:

The patient was discharged to home.

### EXAMPLE 6

A 46 female was admitted to the emergency room with intoxitation and suspected acute kidney dysfunction.

### Case History:

Anorexia nervosa

### Physical Examination:

Weight: 47 Kg
Size: 165 cm
Temperature: 36.5° C.
Heart rate: 80/min
Blood pressure: 76/134 mmHg

### Clinical Signs and Symptoms:

Dyspnea

### Laboratory:

GDF 15 value: 12.78 ng/ml; >8.21 ng/ml (rule in cut off value for admission to hospital).

### Result:

The patient was admitted to hospital.

What is claimed is:

1. A method of identifying if a subject is to be admitted to a hospital, wherein the subject presents to an emergency care unit, a normal care unit, or to an intensive care unit in the hospital, the method comprising the steps of:
determining an amount of growth differentiation factor 15 (GDF 15) in a sample from the subject, and
comparing the amount of GDF 15 determined to a reference amount of GDF 15, whereby the subject is identified as to be admitted to the hospital if the amount of GDF 15 determined is greater than the reference amount of GDF 15.

2. The method according to claim 1, wherein the subject is not suffering from an acute cardiovascular event.

3. The method according to claim 1, wherein the reference amount of GDF 15 is 1200 pg/ml or higher.

4. The method according to claim 1, wherein the reference amount of GDF 15 is 1560 pg/ml or higher.

5. The method according to claim 1, wherein the reference amount of GDF 15 is 8210 pg/ml or higher.

6. A method of predicting a risk of mortality for a subject, wherein the subject presents to an emergency care unit, a normal care unit, or to an intensive care unit of a hospital, comprising the steps of:
determining an amount of growth differentiation factor 15 (GDF 15) in a sample from the subject, and
comparing the amount of GDF 15 determined to a reference amount of GDF 15, whereby the risk of mortality is predicted if the amount of GDF 15 determined is greater than the reference amount of GDF 15.

7. A device for identifying a subject to be admitted to a hospital, comprising a means for determining an amount of GDF 15 in a sample from the subject and a means for comparing said amount to a reference amount of GDF 15.

8. A device for deciding about admitting a subject to a hospital, comprising a means for determining an amount of GDF 15 in a sample from the subject and a means for comparing said amount to a reference amount of GDF 15.

9. A device for predicting a risk of mortality in a subject, comprising a means for determining an amount of GDF 15 in a sample from the subject and a means for comparing said amount to a reference amount of GDF 15.

10. A kit for carrying out the method of claim 1, comprising a means for determining an amount of GDF 15 in a sample from a subject and a means for comparing the amount determined to a reference amount of GDF 15.
